(19) United States
(12) Patent Application Publication
(10) Pub. No.: US 2026/0215678 A1
(43) Pub. Date: Jul. 30, 2026

(54) STEREO ADAPTIVE OPTICS FOR RETINAL VISUALIZATION SYSTEM USING WAVEFRONT SENSING AND CORRECTION

(71) Applicant: Alcon Inc., Fribourg (CH)

(72) Inventor: Steven T. Charles, Memphis, TN (US)

(21) Appl. No.: 19/447,751

(22) Filed: Jan. 13, 2026

Related U.S. Application Data
(60) Provisional application No. 63/751,546, filed on Jan. 30, 2025.

(51) Int. Cl. A61B 3/00; A61B 3/10

(52) U.S. Cl. CPC A61B 3/0058; A61B 3/1015; A61B 2560/0233; A61B 2562/028

(57) ABSTRACT

A retina visualization system includes a laser device operable for outputting a primary laser beam, the laser device including a red, green, blue (RGB) diode array and an infrared (IR) diode, along with a stereo, confocal, biaxial microelectromechanical system (MEMS) scanning system. The scanning system receives the primary laser beam and outputs a scanning laser toward an eye along a visualization path. A Shack Hartmann wavefront sensor proximate the eye senses wavefront distortion in light reflected from the eye. A wavefront control device corrects the wavefront distortion in response to a control signal. A stereo pair of avalanche photodiode (APD) detectors detects light and generates an electronic signal in response thereto. An electronic control unit (ECU) transmits the control signal the wavefront control device to cause the wavefront control device to correct the wavefront distortion into a corrected wavefront, with corrected images displayed via a stereo display device.

### INTRODUCTION

The present disclosure relates to retinal visualization systems and associated methods for assisting in the visualization of inner anatomy of a patient’s eye.

As appreciated in the art, in-office ophthalmic procedures often require a clinician to illuminate and view the retina, macula, and the eye’s vitreous humor. The term “visualization” as used herein refers to the dynamic and interactive examination of the inner eye. The visualized ocular anatomy is generally not for the purpose of digital image capture and analysis during the examination, but rather is viewed and interpreted by the physician in real-time. High image quality with minimal distortion is therefore desirable for accurate diagnosis and treatment.

Vitreoretinal examination is a dynamic process performed with multiple degrees of freedom. During a typical visualization process, a physician constructs a three-dimensional “mental model” of the patient’s inner ocular anatomy. Accurate visualization is highly dependent on a constantly varying vantage point and focal plane. Using a slit lamp, for instance, the physician may manipulate a joystick with a rotating handle to bring target ocular anatomy into and out of focus. The indirect ophthalmoscope for its part is highly dependent on the physician’s head/neck position and orientation, the patient’s ocular rotation and head position, and positioning and angulation of a condensing lens.

Vitreoretinal examination is a stereo, volumetric as opposed to a planar task. This in turn makes stereo/binocular viewing of the ocular anatomy an essential practice. During examination, the physician’s visual cortex calculates depth-of-field by matching corresponding points from each eye located within a volitionally selected depth subset of paired volumes, i.e., the visual horopter or Panum’s fusional area. Near-field and far-field visual noise/scatter/clutter is attenuated outside of the visual horopter. A pair of volumes located in front of and behind the visual horopter are double, but are not seen as such by the physician. Therefore, a need remains for improved spatial awareness, even when the viewed anatomy is not in proper focus.

### SUMMARY

The present disclosure pertain to a stereo adaptive optics system that improves upon the resulting state of the art in terms of image quality during an in-office ophthalmic visualization process. The present adaptive optics-based solutions are intended to provide such improved spatial awareness and other attendant benefits. In contrast to existing adaptive optics systems used for non-real time, non-stereo, monochromatic imaging of individual retinal photoreceptors, primarily in the context of inherited retinal disease, the present approach enables visualization by a clinician.

Magnified views of the inner eye obtained through a patient's dilated pupil may at time be obscured to some extent by corneal, lenticular, or other aberrations. Aberration may be caused, for example, by eye diseases or prior surgeries resulting in corneal asphericity, cataracts, or the presence of an intraocular lens (IOL) implant, any or all of which may lead to an aberrated image when viewed by the physician. Lateral or axial chromatic aberration may be caused by the failure of the eye's optical system or retinal visualization system to properly focus light of assorted colors on the same focal point or plane. Regardless of the root cause, aberration interferes with the physician’s ability to accurately visualize the interior anatomy of the eye. The digital visualization system disclosed herein is therefore configured to correct for such aberration in an in-office environment, using wavefront sensing, thereby improving the quality of digital images presented via stereo, real-time, digital oculars or other display devices.

In particular, embodiments of the present application employ a Shack Hartmann wavefront sensor, e.g., of a type typically used in wavefront-guided LASIK or PRK surgeries, within an office-based visualization system. The adaptive optics system compensate for wavefront distortion in real-time when light is directed into the eye using stereo, confocal, color (i.e., red, green, blue (RGB)), biaxial micro-electromechanical system (MEMS) scanning system. The wavefront sensor is positioned proximate the eye to detect wavefront distortion in light from a scanning laser that is reflected from the eye. An electronic signal representative of the measured distortion is communicated to a processor of an electronic control unit (ECU). The ECU is configured to correct the distorted wavefront, e.g., via one or more wavefront correction algorithms or routines, and to output control signal to a wavefront correction device to cause the wavefront correction device to create a corrected wavefront.

The wavefront control device may include a spatial light modulator (SLM) disposed between the ECU and a pair of avalanche photodiodes. In a possible construction, the SLM includes a liquid crystal on silicon spatial light modulator (LCoS-SLM), or alternatively a transmissive SLM. A deformable mirror is used in other embodiments. Corrected digital image(s) having the corrected wavefront may be displayed on a display device as an end product of the visualization process, e.g., on a stereo, digital display screen, a stereo (3D) heads-up display (HUD), a stereo head mounted display, stereo digital oculars, or any combination thereof.

The present technical solutions, which rely on a narrow bandwidth light source and confocal, MEMS-based spot scanning, help reduce problems typically associated with scattered light from the eye. The disclosed visualization system may also increase a signal-to-noise ratio (SNR), e.g., by about 15 decibels (dB) or more relative to existing in-office visualization systems not using the disclosed confocal approach. Likewise, the present solutions eliminate problematic reflections from the cornea, lens, and other structure of the eye, thereby improving overall results of in-office visualization while providing a clearer and more accurate view of the eye’s anatomy.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is a schematic illustration of an exemplary in-office visualization process that is dynamically performed using a visualization system constructed with adaptive optics and real-time wavefront sensing in accordance with the disclosure.

FIG. 2 is a schematic illustration of a representative embodiment of the adaptive optics system depicted in FIG. 1.

FIG. 3 illustrates a representative Shack Hartmann lenslet array usable as a wavefront sensor within the visualization system of FIG. 2.

FIG. 4 is a flow chart describing a method for visualizing a patient’s eye using the adaptive optics system of FIGS. 1 and 2.

The foregoing and other features of the present disclosure are more fully apparent from the following description and appended claims, taken in conjunction with the accompanying drawings.

### DETAILED DESCRIPTION

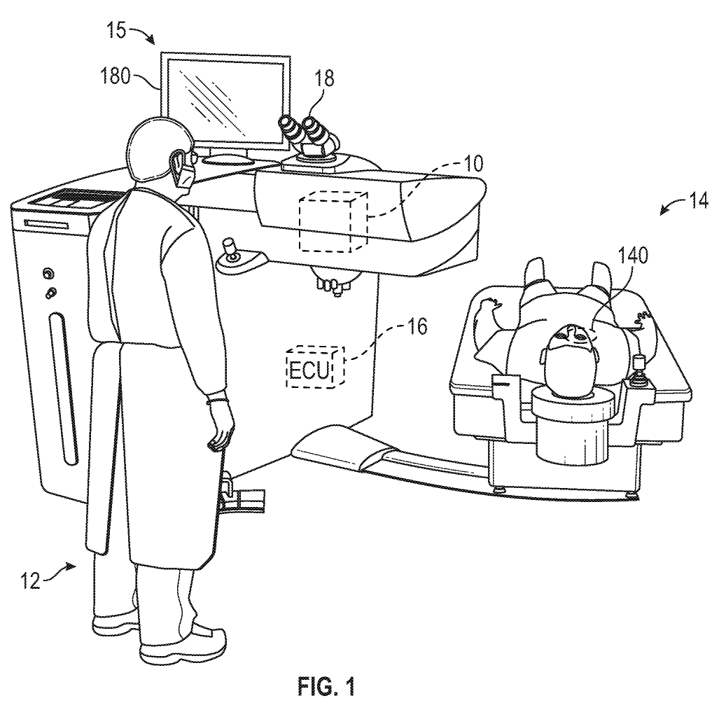

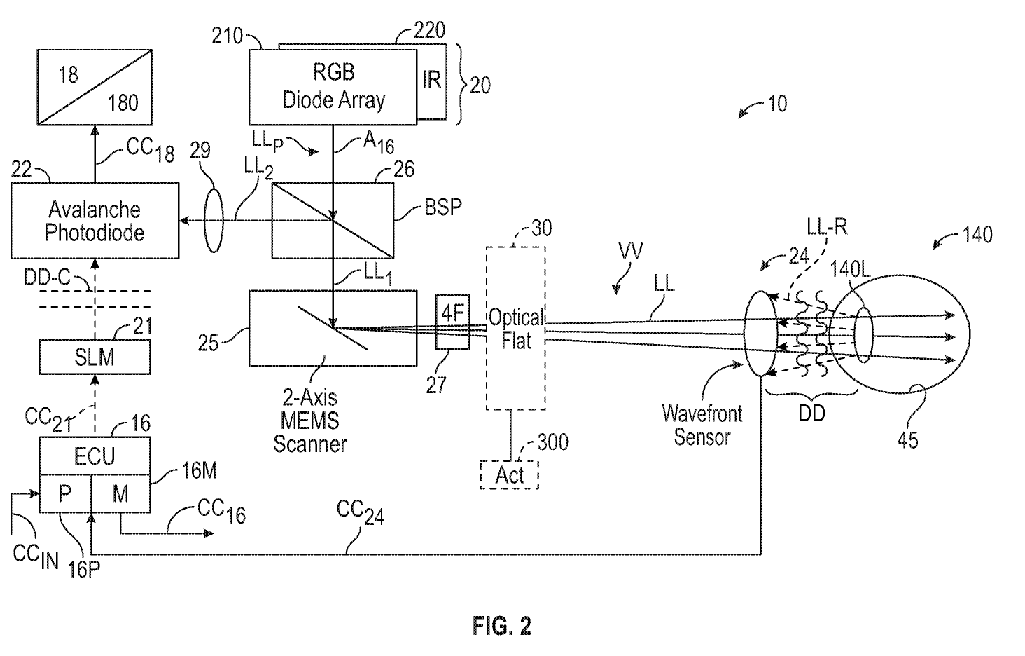

Referring to the drawings, wherein like reference numbers refer to like components, an adaptive optics system 10 is illustrated in FIG. 1 as part of a larger retinal visualization system 15. The adaptive optics system 10 may be used during an in-office examination during which a clinician 12 visualizes an eye 140 of a patient 14. The adaptive optics system 10 includes or is in communication with an electronic control unit (ECU) 16. The ECU 16 is operable for performing a confocal, biaxial MEMS-based spot scan of the eye 140 using a laser device 20, a MEMS scanning system 25, a 4F optical system 27, and a stereo pair of APD detectors 22 as shown in FIG. 2. Digital images of the eye 140 are collected and presented using the present approach with improved image resolution, color rendition, and dynamic range compared to digital images of the eye 140 captured without benefit of the adaptive optics system 10.

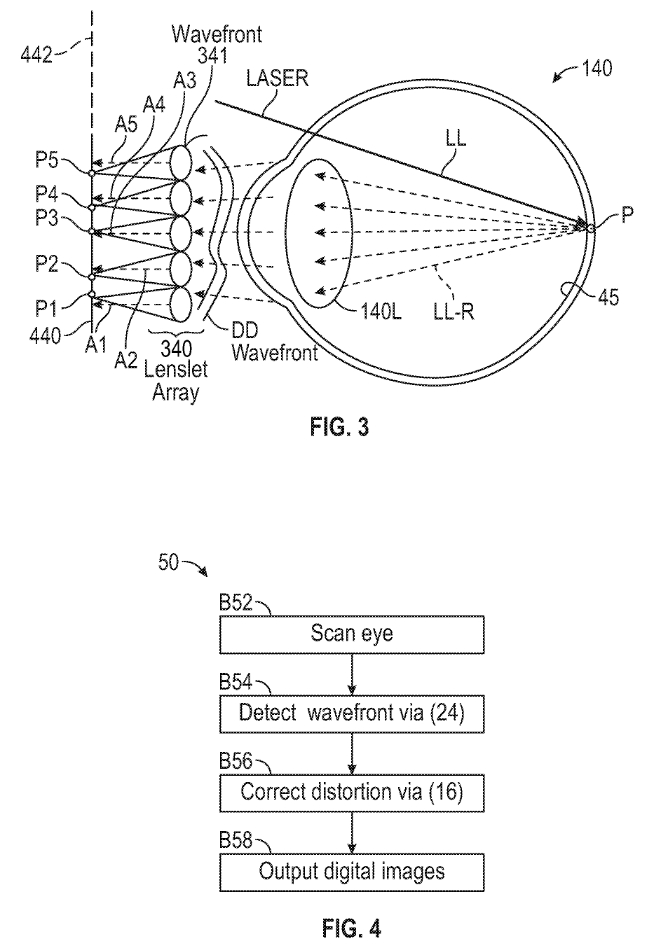

In a possible use scenario, the clinician 12 of FIG. 1 may view digital images of the eye 140 on a microscope integrated display (MID) or digital oculars 18, e.g., organic light-emitting diode (OLED) binoculars, and/or one or more display devices 180 operable for displaying digital images of the eye 140 with a corrected wavefront (DD-C) as set forth herein. Digital images of the eye 140 as described herein include one or more distorted wavefronts of light from a scanning laser (LL of FIG. 2) reflected off an interior of the eye 140 and thereafter fully or partially corrected via the adaptive optics system 10 as set forth below. A representative construction of the adaptive optics system 10 is described in detail below with particular reference to FIG. 2, with an exemplary Shack Hartmann construction of a wavefront sensor shown in FIG. 3. An exemplary method 50 for using the adaptive optics system 10 of FIGS. 1 and 2 is illustrated in FIG. 4.

Referring to FIG. 2, enhanced visualization capabilities are provided herein using stereo, confocal, biaxial microelectromechanical sensor (MEMS)-based scanning of the eye 140 in a clinical use/visualization setting, i.e., during performance of a non-surgical task. In particular, the adaptive optics system 10 employs a wavefront sensor 24 proximate the eye 140, e.g., a Shack Hartmann sensor as described in more detail with reference to FIG. 3, or an interferometer or another suitable sensor configured to sense wavefront distortion in light that is reflected from the eye 140. The wavefront sensor 24 for its part is used in conjunction with the ECU 16 in lieu of, e.g., the use of surgical tools as guide stars as set forth in United States Patent No. 11,883,096B2, which issued on Jan. 30, 2024, which is hereby incorporated by reference in its entirety. The specific modifications and adaptations described herein thus extend the benefits of adaptive optics to in-office retinal visualization.

Referring briefly to FIG. 3, a representative implementation of the wavefront sensor 24 of FIG. 2 includes a Shack Hartmann wavefront sensor 240 having a lenslet array 340 and a photosensor 440, the latter of which is arranged in an image plane 442. When the scanning laser (LL) enters the eye 140 and contacts the retina 45 at point P, reflected light (LL-R) of the scanning laser (LL) reflects from the retina 45 and passes through the lens 140L, i.e., a natural lens or an IOL. Wavefront distortion (DD) is sensed by the lenslet array 340. The lenslet array 340, which forms the core of the wavefront sensor 24, includes a plurality of miniature lenses (“lenslets”) 341 arranged in a grid. As the distorted wavefront falls incident upon the lenslet array 340, the individual lenslets 341 sample a local portion of the distorted wavefront and focus light as spots (e.g., P1, P2 P3, P4, P5) on the image plane 442 of the photosensor 440. Local slope of the distorted wavefront is then determined as displacement of each of the spots from a respective center axis (e.g., A1, A2, A3, A4, A5) of the lenslets 341.

The ECU 16 of FIGS. 1 and 2, which is in communication with the wavefront sensor 24, is configured to receive an electronic signal (CC₂₄) from the wavefront sensor 24 indicative or representative of the sensed wavefront distortion of the distorted wavefront (DD). The ECU 16 then reconstructs the wavefront distortion in logic, and transmits a control signal (CC₂₁) to a wavefront control device 21 (FIG. 2) to cause the wavefront control device 21 to correct the wavefront distortion into a corrected wavefront (DD-C). This action ultimately enables the production of digital images with improved image quality, e.g., by eliminating corneal and lenticular/IOL aberration as noted above.

Referring once again to FIG. 2, the adaptive optics system 10 includes the laser device 20 operable for outputting a primary laser beam (LL_{P}). The laser device 20 may include a color/red, green, and blue (RGB) laser diode array 210 and an infrared (IR) laser diode 220 in a possible embodiment. In one or more implementations, the laser device 20 is selectively switchable between an RGB laser beam from the RGB laser diode array 210 and an IR laser beam from the IR laser diode 220. The adaptive optics system 10 also includes the wavefront control device 21, e.g., a spatial light modulator (SLM) 21 or other device as set forth below, and the stereo pair of avalanche photodiodes 22. Additionally, the adaptive optics system 10 in one or more embodiments also includes the confocal, biaxial (dual-axis) MEMS scanning system 25 configured to receive the primary laser beam (LL_{P}) from the laser device 20 and output the scanning laser (LL) towards the eye 140 along a visualization path (VV), i.e., orthogonal to a beam path (A₁₆) of the primary laser beam (LL_{P}).

As part of the present approach, the ECU 16 of FIG. 2 is configured to control motion, position, state, and/or other parameters of the various components of the adaptive visualization system 10. The ECU 16 is operable for communicating with the various components of the adaptive optics system 10 as noted above when performing a confocal spot scan of the eye 140, with wavefront distortion correction performed via the wavefront sensor 24 and wavefront correction device 21 as set forth herein.

The confocal, biaxial MEMS scanning system 25 of FIG. 2 may be embodied as a dual-axis piezoelectric scanning device having a resonant horizontal axis (“fast axis”) and a quasi-static vertical axis (“slow axis”). For example, the MEMS scanning system 25 could have individually-controllable turning mirrors or prisms (not shown) to provide a mechanically-variable parallax angle for the clinician 12 of FIG. 1. In other possible configurations, the MEMS scanning system 25 may be implemented as one or more electromagnetic or electrostatic devices. Commercially-available options for the MEMS scanning system 25 include, by way of an example, a MEMS-based laser scanning module from Microvision, Inc., of Redwood, WA. Such RGB laser diode-based embodiments as contemplated herein are highly compact with superior dynamic range, among other attendant benefits.

With respect to the laser device 20 of FIG. 2, this collimated light-emitting component may include three or more laser diodes collectively forming the RGB laser diode array 210 and the IR laser diode 220. Commercially-available, highly compact RGB laser modules may be used for this purpose, e.g., the Veglas^{TM} RGB laser module from ams OSRAM AG. In an optional embodiment, the light source 20 is selectively switchable between constituent red, green, and blue color laser diodes and the IR laser diode 220. The laser device 20 may be implemented as a Fabry-Pérot laser diode setup with a relatively narrow bandwidth spectrum, with control degrees of freedom in terms of gain and feedback within a laser cavity. As will be appreciated by those skilled in the art, unlike visible light, near IR (NIR) light does not require a darkened room or pupillary dilation, e.g., via dilation drops. For children and other patients with photophobia, the NIR spectrum may be a useful alternative or a complementary light source for direct viewing during examination. NIR implementation alone could be used in other embodiments. Such an approach may be simpler in terms of required optics, and would eliminate the need for multiple mirrors /beam splitters, but at the expense of additional electronic sensing and display capabilities, as appreciated in the art.

The present “RGB + IR” solutions in conjunction with the adaptive optics system 10 could be used to replace indirect or slit lamp light sources with the IR laser diode 220 (about 820 nm) and the RGB laser diode array 210, i.e., white light, in a switchable configuration. That is, the clinician 12 could turn the IR laser diode 220 on and off when needed, with the stereo pair of APDs 22 being sensitive enough to detect such light and generate an output signal 220, the latter ultimately causing the digital oculars 18 or display device 180 to display digital images of the eye 140. However, as IR direct-view confocal visualization is not possible, IR-based implementations contemplated herein may require the 4F optical system 27 for true confocal capability, along with the laser device 20 and the digital oculars 18 (FIG. 1), or variations thereof. Images from IR illumination in some implementations could be displayed as green (about 550 nm) on a black background.

In the representative implementation of FIG. 1, the primary laser beam (LL_{P}) is emitted by the laser device 20 and directed along the beam path (A₁₆) toward a beam splitter (BSP) 26, possibly via a condensing lens (not shown) likewise arranged in the beam path (A₁₆). The beam splitter 26 located in this position divides the primary laser beam (LL_{P}) into respective first and second beam portions (LL₁ and LL₂). The beam splitter 26 may be arranged adjacent to a focusing lens (not shown) that is configured to direct the second beam portion (LL₂) through a pinhole (not shown) and onto the stereo pair of avalanche photodiode (APD) detectors 22. The APD detector 22 for its part is in communication with the ECU 16, either wirelessly or via physical transfer conductors, and thus provides a sensory feedback signal to the ECU 16 as part of the ongoing visualization process. Reflected light (LL-R) from the eye 140 is also directed to the APD detector 22 as part of the detection process for ultimate viewing by the clinician 12 of FIG. 1, with wavefront distortion 30 in such reflected light (LL-R) detected by sensor 24 and reported to the ECU 16 as part of the present strategy.

The beam splitter 26 of FIG. 2 is thus positioned and equipped to direct the first beam portion (LL₁) from the laser device 20 into the MEMS scanning system 25. The MEMS scanner 20 thereafter oscillates and directs the first beam portion (LL₁) as a scanning laser (LL) into a 4F optical system 27. A representative construction of the 4F system 27 is described in US Patent Application Serial No. 18/462,767, now published as US Publication No. 2024/0138675A1, which was filed on Sep. 7, 2023, and which is hereby incorporated by reference in its entirety. The ECU 16 is thus configured to control a confocal spot scanning process of the eye 140 during by controlling the output of the scanning laser (LL) and the associated components of the depicted adaptive optics system 10.

The avalance photodiode (APD) detector 360 (steeo pair of avalanche photodiodes) as shown schematically in FIG. 2 is used herein with the laser device 20 to provide spot scanning and confocal imaging capabilities within the scope of the disclosure, while largely eliminating scattered light. As appreciated by those of ordinary skill in the art, APDs are a particular type of photodiode in which high internal gain is produced by applying a reverse voltage. The resulting high gain, sensitivity, and fast response times of the APD detector 360 thus increase SNR relative to other types of photodetectors, and lends itself to the switchable color-IR technique described herein. Exemplary commercially-available APD detectors 360 usable herein include those offered by Hamamatsu Photonics K.K. of Hamamatsu City, Japan, or Excelitas Technologies^{®} Corporation of Waltham, Massachusetts, USA.

As appreciated by those skilled in the art, time-sequential RGB images are integrated by the human brain within the brain’s visual cortex, provided the frame rate for each perceived color exceeds a critical flicker fusion rate of at least 60 Hz per color, or about 180 Hz in the aggregate for RGB applications. Confocal spot scanning as contemplated herein, and as enabled by the 4F optical system 27 and the stereo pair of APD detectors 22, can improve the SNR by reducing scattered light. As appreciated, the 4F optical system 27 places a spatial filter, i.e., a pinhole, in the Fourier plane for true confocal scanning. However, confocal spot scanning significantly decreases the depth-of-field perceived by the clinician 12 of FIG. 1.

As part of the illustrated configuration, therefore, an optional liquid lens 29 may be situated adjacent to the APD detectors 22. The ECU 16 in such an embodiment could drive the liquid lens 29 with a sine wave as part of the control signals (CC₁₆) to increase the depth-of-field of the clinician 12 in stereo, RGB laser diode spot, or slit scanning embodiments of the automated visualization system 15, with limited degrees of freedom. A sine wave-driven implementation could help reduce speckle in a direct-view, stereo RGB laser diode spot or slit scanning embodiment of the adaptive optics system 10 The liquid lens 29, e.g., from Optotune Switzerland, AG, can therefore provide the desirable depth-of-field, and can be selectively driven through a large amplitude focus range, typically in about 1-2 milliseconds (ms).

Although the ECU 16 is depicted schematically in FIG. 2 as a unitary box for illustrative clarity and simplicity, the ECU 16 could include one or more networked devices each with a central processing unit or other processor 16P and sufficient amounts of memory 16M, including a non-transitory (e.g., tangible) medium that participates in providing data/instructions that may be read by the processor 16P. The memory 16M may take many forms, including but not limited to non-volatile media and volatile media. As will be appreciated, non-volatile media may include optical and/or magnetic disks or other persistent memory, while volatile media may include dynamic random-access memory (DRAM), static RAM (SRAM), etc., any or all which may constitute a main memory of the ECU 16. Input/output circuitry may be used to facilitate connection to and communication with the various peripheral devices used during the ophthalmic procedure. Other hardware not depicted but commonly used in the art may be included as part of the ECU 16, including but not limited to a local oscillator or high-speed clock, signal buffers, filters, etc. Connectivity may be provided via BLUETOOTH, Wi-Fi, HDMI, NFC, DISPLAY PORT, THUNDERBOLT, etc.

Still referring to the adaptive optics system 10 of FIG. 2, while embodiments foregoing use of an optional optical flat 30 are optimal in the present use cases by providing separate channels for stereo viewing, the optical flat 30 may be used in one or more alternative constructions. The optional optical flat 30 may be useful in certain embodiments in which the laser device 20, pinhole (not shown), and the stereo pair of APD detectors 22 are shared components, i.e., a single-channel implementation. The optical flat 30 is configured to oscillate around its vertical axis when driven by an actuator 300, e.g., a resonant moving magnet galvometer (galvo), a piezoelectric stack (piezo-actuator), or another application-suitable oscillatory actuation device. As appreciated in the art, the vertical axis, which is relatively slow and non-resonant, is position-driven, while the horizontal axis is fast, thus requiring a resonant galvo.

The optional optical flat 30 in one or more embodiments is configured as a polished flat reference surface, such as fused silica or another application-suitable material. In the contemplated construction of the adaptive optics system 10, for instance, the optical flat 30 is configured to oscillate on the vertical axis in a scan/de-scan path of the MEMS scanner 25 so as to achieve stereo parallax. That is, a parallax shift of a type required for proper stereo viewing by the clinician 12 of FIG. 1 is provided by operation of the optical flat 30 as the optical flat 30 oscillates around its vertical axis. To this end, the actuator 300 when attached to the optical flat 30 is configured to oscillate the optical flat 30 at a calibrated frequency, which as used herein should be greater than an aggregate frame rate of at least 60Hz per color or 180Hz for the illustrated RGB embodiment of FIG. 2. Thus, the scanning laser (LL) passes through the optical flat 30 and enters the eye 140 through a lens 140L (FIG. 3) thereof, and thereafter illuminates the retina 45 or posterior wall of the eye 140 within a controlled scanning range.

WAVEFRONT CORRECTION: the adaptive optics system 10 is used to correct the sensed wavefront distortion (DD) to a corrected wavefront (DD-C) using the ECU 16 and wavefront correction device 21, thereby improving image resolution. To that end, the ECU 16 is programmed to control and correct the phase of the wavefront distortion (DD). As part of this approach, the wavefront correction device 21 may be variously implemented as an SLM, an LCoS-SLM, a transmissive SLM, or a deformable mirror. Wavefront distortion (DD) of visible and/or IR light delivered by the laser device 20 and reflected by the interior anatomy of the eye 140 as shown in FIG. 3 may be detected by the wavefront sensor 24 and automatically corrected by operation of the ECU 16 and the wavefront correction device 21.

As appreciated in the art, wavefront distortion (DD) may be caused by cornea of the eye 140, or the lens 140L, e.g., having a cataract thereon, or by an intraocular lens (IOL), etc., after light is reflected from the eye 140 as reflected light (LL-R). Such reflected light (LL-R) may be also from the retina, sclera, choroid, vitreous, scar tissue, epiretinal membrane, internal limiting membrane, etc. The ECU 16 may use different wavefront shaping for each of the three light colors red, green and blue (or IR), which may reduce axial and lateral chromatic aberration in the adaptive optics system 10. Using its processor 16P, the ECU 16 may execute instructions to produce a digital image in which at least one wavefront distortion in the light detected by the APD detectors 22 is partially or fully corrected.

Referring to FIG. 4, method 50 is described in terms of algorithm code segments or logic blocks. Each block is executable by the processor 16P to cause the ECU 16 of FIGS. 1 and 2 to perform the indicated steps. Beginning with block B52, the method 50 includes performing confocal, MEMS-based spot scanning of the eye 140 with the scanning laser (LL) of FIG. 2. Scanning as contemplated herein involves the use of the RGB laser diode array 210 and IR laser diode 220 of FIG. 2, possibly switching between the two devices as noted above, with confocal scanning microscopy using the dual axis MEMS scanning system 25, 4F optical system 27, and optional optical flat 30. The method 50 proceeds to block B54 once scanning of the eye 140 commences.

At block B54, the method 50 includes detecting the wavefront distortion (DD) of FIGS. 2 and 3 via the wavefront sensor 24. Wavefront sensing may be performed in one or more embodiments using a Shack Hartmann sensor shown in FIG. 3 as noted above, an interferometer, or using another suitable sensor or sensors. An electronic signal (CC₂₄) indicative of the sensed wavefront distortion (DD) is then communicated to the ECU 16, either wirelessly or over physical transfer conductors. The method 500 thereafter proceeds to block B56.

Block B56 of FIG. 4 includes correcting the sensed distortion via the ECU 16. For instance, the ECU 16 may be configured to correct wavefront distortion using a wavefront shaping algorithm to reduce axial and lateral chromatic aberration. Wavefront correction may use corrective wavefront shaping to individually correct reflected red, green, and blue wavelengths of light and ensure the light of various colors is focused on the same convergence point or image plane. The wavefront control device 21 of FIG. 2 for its part may be responsive to the control signals (CC₂₁) to implement region of interest gain control and improve dynamic range. In one or more embodiments, the wavefront control device 21 may be constructed as an SLM or LCoS device, or alternatively as a transmissive SLM or a deformable mirror. The method 50 proceeds to block B58 once the wavefront distortion (DD) has been processed into the corrected wavefront (DD-C) and the wavefront control device 21 has been controlled to generate the corrected wavefront (DD-C).

Block B58 entails outputting one or more digital images to the digital oculars 18 or display device 180 of FIG. 1, e.g., via an output signal 220 from the stereo pair of avalanche photodiode (APD) detectors 22 of FIG. 2. The clinician 12 is thereafter afforded a corrected view of the eye 140 during the in-office examination.

The present teachings provide improved image quality, particularly of corneal and lenticular/IOL aberration, with visualization assisted by the confocal MEMS scanning approach and real-time use of wavefront sensing. The solutions described herein reduce higher order aberrations, defocus, and effects of astigmatism and axial and lateral chromatic aberration. Use of the adaptive optic system 10 also reduces photophobia and pupillary dilation in some in-office retinal visualization scenarios by using infrared (IR) illumination as an option. Additionally, the use of confocal scanning via the setup of FIG. 2, i.e., its biaxial scanning laser diode point source, 4F optics, spatial filter, and avalanche photodiode sensors, is intended to reduce scattered light from cataracts, IOLs, posterior capsular opacification (PCO), etc. These and other attendant benefits will be readily appreciated by those skilled in the art in view of the forgoing disclosure.

Embodiments of the present disclosure are described herein. It is to be understood, however, that the disclosed embodiments are merely examples and other embodiments can take various and alternative forms. The Figures are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the present disclosure. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar importance.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

1. A retina visualization system, comprising:
a laser device operable for outputting a primary laser beam;
a stereo, confocal, biaxial microelectromechanical system (MEMS) scanning system configured to receive the primary laser beam from the laser device, and to output a scanning laser toward an eye along a visualization path through a 4F optical system arranged in the visualization path;
a wavefront sensor positioned proximate the eye and configured to sense wavefront distortion in light from the scanning laser that is reflected by the eye;
a wavefront control device operable for correcting the wavefront distortion in response to a control signal;
a stereo pair of avalanche photodiode (APD) detectors operable to detect light with a corrected wavefront and generate an output signal to a display device in response thereto; and
an electronic control unit (ECU) in communication with the wavefront sensor and the APD detectors, wherein the ECU is configured to receive an electronic signal from the wavefront sensor indicative of the wavefront distortion, and to transmit the control signal the wavefront control device to cause the wavefront control device to correct the wavefront distortion into the corrected wavefront.

2. The retina visualization system of claim 1, wherein the wavefront sensor includes a Shack Hartmann sensor.

3. The retina visualization system of claim 1, wherein the wavefront sensor includes an interferometer.

4. The retina visualization system of claim 1, wherein the laser device includes a red, green, blue (RGB) laser diode array and an IR laser diode.

5. The retina visualization system of claim 4, wherein the laser device is selectively switchable between an RGB laser beam from the RGB laser diode array and IR laser beam from the IR laser diode.

6. The retina visualization system of claim 1, wherein the wavefront control device comprises a spatial light modulator (SLM).

7. The retina visualization system of claim 5, wherein the SLM comprises a liquid crystal on silicon spatial light modulator (LCoS-SLM).

8. The retina visualization system of claim 1, wherein the wavefront control device comprises a deformable mirror.

9. The retina visualization system of claim 1, further comprising the display device, wherein the display device includes digital oculars operable for displaying stereo images of the eye with the corrected wavefront.

10. The retina visualization system of claim 9, wherein the digital oculars are organic light-emitting diode (OLED) binoculars.

11. The retina visualization system of claim 1, wherein the ECU is operable for performing a confocal spot scan of the eye using the light source, the MEMS scanner, the 4F optical system, and the stereo pair of APD detectors.

12. A retina visualization method, comprising:
outputting a primary laser beam via a laser device, including outputting a color laser beam via a red, green, blue (RGB) laser diode array and outputting an infrared (IR) laser beam via an IR laser diode;
receiving the primary laser beam via a stereo, confocal, biaxial microelectromechanical system (MEMS) scanning system;
in response to receiving the primary laser beam, outputting a scanning laser via the MEMS scanning system toward an eye along a visualization path, including
directing the scanning laser through a 4F optical system arranged along the visualization path;
using a wavefront sensor positioned proximate the eye to sense wavefront distortion in light from the scanning laser that is reflected by the eye;
correcting the wavefront distortion via a wavefront control device in response to a control signal;
detecting light with a corrected wavefront using a stereo pair of avalanche photodiode (APD) detectors;
generating an output signal to a display device, via the stereo pair of APD detectors, in response to detecting the light;
receiving an electronic signal from the wavefront sensor via an electronic control unit (ECU), the electronic signal being representative of the wavefront distortion; and
transmitting the control signal the wavefront control device via the ECU to cause the wavefront control device to correct the wavefront distortion into the corrected wavefront.

13. The retina visualization method of claim 12, wherein using the wavefront sensor positioned proximate the eye to sense the wavefront distortion includes using a Shack Hartmann sensor.

14. The retina visualization method of claim 12, wherein using the wavefront sensor positioned proximate the eye to sense the wavefront distortion includes using an interferometer.

15. The retina visualization method of claim 12, wherein correcting the wavefront distortion via the wavefront control device includes using a spatial light modulator (SLM).

16. The retina visualization method of claim 15, wherein using the SLM comprises using a liquid crystal on silicon spatial light modulator (LCoS-SLM).

17. The retina visualization method of claim 12, wherein correcting the wavefront distortion via the wavefront control device includes using a deformable mirror.

18. The retina visualization method of claim 12, further comprising:
using the laser device to selectively switch between an RGB laser beam from the RGB laser diode array and an IR laser beam from the IR laser diode.

19. The retina visualization method of claim 12, further comprising:
displaying digital images of the eye with the corrected wavefront via a display device.

20. A retina visualization system, comprising:
a laser device operable for outputting a primary laser beam, the laser device including a red, green, blue (RGB) laser diode array and an infrared (IR) laser diode;
a stereo, confocal, biaxial microelectromechanical system (MEMS) scanning system configured to receive the primary laser beam and output a scanning laser toward an eye along a visualization path through 4F optical system arranged along the visualization path;
a Shack Hartmann wavefront sensor positioned proximate the eye and configured to sense wavefront distortion in light that is reflected from the eye;
a silicon spatial light modulator (LCoS-SLM) operable for correcting the wavefront distortion in response to a control signal;
a pair of avalanche photodiode (APD) detectors operable to detect light and generate an output signal in response thereto;
an electronic control unit (ECU) in communication with the wavefront sensor, wherein the ECU is configured to receive an electronic signal from the wavefront sensor representative of the wavefront distortion, and transmit the control signal the wavefront control device to cause the wavefront control device to correct the wavefront distortion into a corrected wavefront; and
digital oculars operable for displaying digital images of the eye with the corrected wavefront in response to the output signal from the APD detectors.
